(19) United States
(12) Patent Application Publication
(10) Pub. No.: US 2009/0222098 A1
(43) Pub. Date: Sep. 3, 2009

(54) Spinal nucleus replacement with varying modulus

(75) Inventors: Hai H. Trieu, Cordova, TN (US); Michael C. Sherman, Memphis, TN (US); Mingyan Liu, Bourg la Reine (FR)

Correspondence Address: MEDTRONIC;Attn: Noreen Johnson - IP Legal Department, 2600 Sofamor Danek Drive, MEMPHIS, TN, 38132, US

(73) Assignee: Warsaw Orthopedics, Inc., Warsaw, IN (US)

(21) Appl. No.: 12/038,992

(22) Filed: Feb. 28, 2008

(51) Int. Cl. A61F 2/44

(52) U.S. Cl. 623/17.16; 623/17.12

(57) ABSTRACT

An intervertebral disc augmentation implant comprises an implant body adapted for implantation within an annulus fibrosis the intervertebral disc, adjacent to an at least partially intact nucleus pulposus of the intervertebral disc, and comprising a core area, a peripheral wall area, a top face area, and a bottom face area. The implant body is formed from at least one material and the implant body has a modulus of elasticity gradient that gradually changes from the core area of the implant body to the peripheral wall area of the implant body.

### BACKGROUND

Within the spine, the intervertebral disc functions to stabilize and distribute forces between vertebral bodies. The intervertebral disc comprises a nucleus pulposus which is surrounded and confined by the annulus fibrosis.

Intervertebral discs are prone to injury and degeneration. For example, herniated discs typically occur when normal wear, or exceptional strain, causes a disc to rupture. Degenerative disc disease typically results from the normal aging process, in which the tissue gradually loses its natural water and elasticity, causing the degenerated disc to shrink and possibly rupture.

Intervertebral disc injuries and degeneration may be treated by fusion of adjacent vertebral bodies or by replacing the intervertebral disc with a prosthetic. To maintain as much of the natural tissue as possible, the nucleus pulposus may be supplemented or replaced while maintaining all or a portion of the annulus. A need exists for nucleus replacement and supplementation implants that will reduce the potential for implant migration within the annulus and/or expulsion from the annulus.

### SUMMARY

In one embodiment, an intervertebral disc augmentation implant comprises an implant body adapted for implantation within an annulus fibrosis the intervertebral disc, adjacent to an at least partially intact nucleus pulposus of the intervertebral disc, and comprising a core area, a peripheral wall area, a top face area, and a bottom face area. The implant body is formed from at least one material and the implant body has a modulus of elasticity gradient that gradually changes from the core area of the implant body to the peripheral wall area of the implant body.

In another embodiment, a method of augmenting a nucleus pulposus comprises accessing an annulus surrounding the nucleus pulposus and forming an opening in the annulus. The method further comprises inserting an intervertebral nucleus augmentation implant. The implant comprises an implant body including a core area, a peripheral wall area, a top face, and a bottom face. The implant body is formed from at least one material and the implant body has a modulus of elasticity gradation that gradually changes from the core area of the implant body to the peripheral wall area of the implant body.

In another embodiment, an implant for augmenting or replacing the natural nucleus pulposus within an intervertebral space comprises a central region and an outer region extending between the central region and an outer wall. The outer region has a modulus of elasticity that becomes progressively higher from the central region to the outer wall. The implant is sized for insertion through an opening in an annulus fibrosis surrounding the nucleus pulposus.

Additional embodiments are included in the attached drawings and the description provided below.

### BRIEF DESCRIPTION OF THE DRAWINGS

FIG. 1 is a sagittal view of a section of a vertebral column.

FIGS. 2 and 3 are a cross-sectional side view and a top view, respectively, of a nucleus augmentation implant having regions of different moduli of elasticity.

FIGS. 4 and 5 are a cross-sectional side view and a top view, respectively, of another nucleus augmentation implant having regions of different moduli of elasticity.

FIGS. 6 and 7 are a cross-sectional side view and a top view, respectively, of another nucleus augmentation implant having regions of different moduli of elasticity.

FIGS. 8 and 9 are a cross-sectional side view and a top view, respectively, of another nucleus augmentation implant having regions of different moduli of elasticity.

FIGS. 10-15 are cross-sectional side views of nucleus augmentation implants with gradual gradation in the modulus of elasticity from the center of the implant toward the periphery of the implant.

FIG. 16 is a top view of the nucleus augmentation implant of FIG. 14.

### DETAILED DESCRIPTION

The present disclosure relates generally to devices and methods for relieving disc degeneration or injury, and more particularly, to devices and methods for augmenting a nucleus pulposus. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

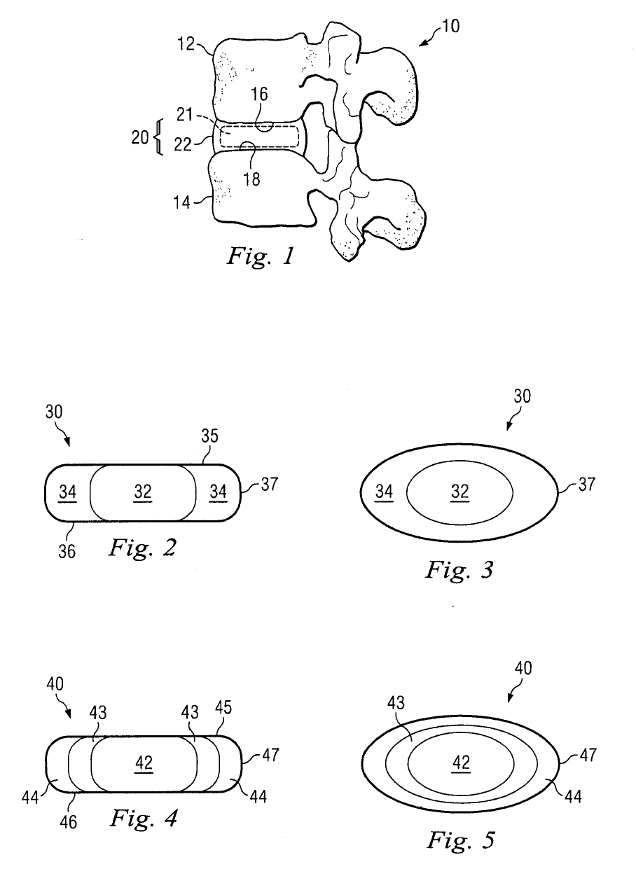

Referring first to FIG. 1, the reference numeral 10 refers to a vertebral joint section or a motion segment of a vertebral column. The joint section 10 includes adjacent vertebral bodies 12, 14. The vertebral bodies 12, 14 include endplates 16, 18, respectively. An intervertebral disc space 20 is located between the endplates 16, 18, and an annulus fibrosis 22 surrounds the space 20. In a healthy joint, the space 20 contains a nucleus pulposus 21. The nucleus pulposus 21 may degenerate with age, disease, or trauma.

Referring now to FIGS. 2 and 3, a nucleus augmentation implant 30 may be used to supplement the function of an existing nucleus pulposus or replace all or a portion of the nucleus pulposus. Thus, the implant 30 may fill all or a portion of the disc space 20 within the annulus 22. The implant 30 comprises two regions, a central region 32 and a peripheral region 34. The implant has a top face 35, a bottom face 36, and a side wall 37. Each region 32, 34 has a different modulus of elasticity. For example, central region 32 may have a lower modulus of elasticity than the peripheral region 34, such that the implant 30 has a softer center and a stiffer peripheral area. In alternative embodiments, the inverse may be true with the center stiffer than the peripheral area. In this embodiment, at least a portion of the central region 32 extends from the top face 35 of the implant 30 to the bottom face 36 with the modulus of elasticity varying primarily from the center of the implant toward the side wall 37.

Referring now to FIGS. 4 and 5, a nucleus augmentation implant 40 may be used to supplement the function of an existing nucleus pulposus or replace all or a portion of the nucleus pulposus. Thus, the implant 40 may fill all or a portion of the disc space 20 within the annulus 22. The implant 40 comprises three regions, a central region 42, a middle region 43, and a peripheral region 44. The implant has a top face 45, a bottom face 46, and a side wall 47. Each region 42, 43, 44 has a different modulus of elasticity. For example, central region 42 may have a lower modulus of elasticity than regions 43, 44, and the middle region 43 may have a lower modulus of elasticity than the region 44, such that the implant 40 has a softer center and an increasingly stiff outer area. In alternative embodiments, the inverse may be true with the center stiffer than the peripheral area. In this embodiment, at least a portion of the central region 42 extends from the top face 45 of the implant 40 to the bottom face 46 with the modulus of elasticity varying primarily from the center of the implant toward the side wall 47.

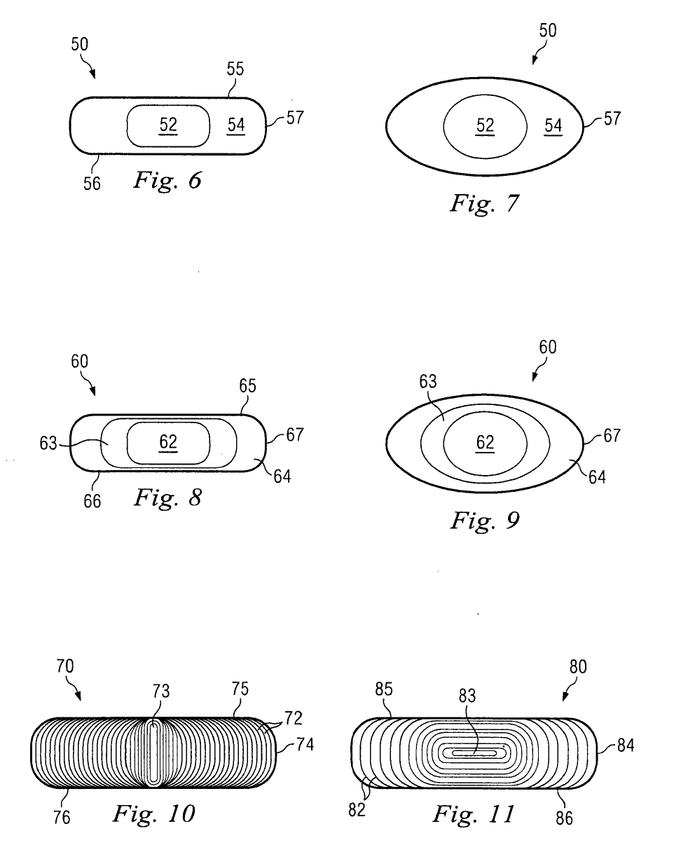

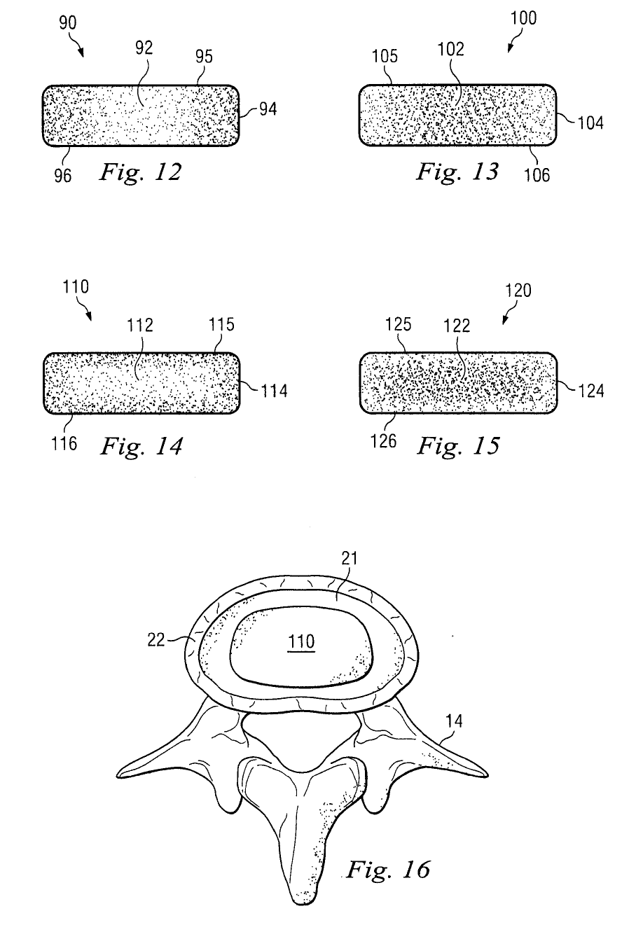

Referring now to FIGS. 6 and 7, a nucleus augmentation implant 50 may be used to supplement the function of an existing nucleus pulposus or replace all or a portion of the nucleus pulposus. Thus, the implant 50 may fill all or a portion of the disc space 20 within the annulus 22. The implant 50 comprises two regions, a central region 52 and a peripheral region 54. The implant has a top face 55, a bottom face 56, and a side wall 57. Each region 52, 54 has a different modulus of elasticity. For example, central region 52 may have a lower modulus of elasticity than region 54, such that the implant 50 has a softer center and a stiffer peripheral area. In alternative embodiments, the inverse may be true with the center stiffer than the peripheral area. In this embodiment, the central region 52 is encased within the peripheral region 54 with the region 54 extending between the central region 52 and the top and bottom faces 55, 56. The modulus of elasticity varies both from the center of the implant toward the side wall 57 and from the center toward the top and bottom faces 55, 56.

Referring now to FIGS. 8 and 9, a nucleus augmentation implant 60 may be used to supplement the function of an existing nucleus pulposus or replace all or a portion of the nucleus pulposus. Thus, the implant 60 may fill all or a portion of the disc space 20 within the annulus 22. The implant 60 comprises three regions, a central region 62, middle region 63, and a peripheral region 64. The implant has a top face 65, a bottom face 66, and a side wall 67. Each region 62, 63, 64 has a different modulus of elasticity. For example, central region 62 may have a lower modulus of elasticity than regions 63, 64 and middle region 63 may have a lower modulus of elasticity than region 64. The resulting implant 60 has a softer center and a stiffer peripheral area. In alternative embodiments, the inverse may be true with the center stiffer than the peripheral area. In this embodiment, the central region 62 is encased within the middle region 63 and the middle region 63 encased within the peripheral region 64. The middle region 63 and the peripheral region 64 extend between the central region 62 and the top and bottom faces 65, 66. The modulus of elasticity varies both from the center of the implant toward the side wall 67 and from the center toward the top and bottom faces 65, 66.

Referring now to FIG. 10, an implant 70 includes a series of thin layers 72 emanating from a central area 73 and extending outward toward a side wall 74. The implant 70 has a top face 75 and a bottom face 76. The layers 72 may be concentric about the central area 73 and each layer may extend substantially from the top face 75 to the bottom face 76. The layers 72 may have different moduli of elasticity. For example, the modulus of elasticity of the central area 73 may be relatively low, with the moduli of the layers growing increasingly larger toward the outer layers near the side wall 74. In this configuration, the implant 70 would have a soft center and an increasingly stiff periphery. In one embodiment, five or more thin layers may be used.

When the implant 70 is subjected to an asymmetric load, the variance in modulus from the central area 73 and across the layers 72 may allow the central area to shift away from the load, thereby reducing the likelihood that the entire implant will migrate or become expelled from the annulus 22. The thin layers 72 may also affect a relatively smooth, less abrupt, change in moduli which may permit an even, continuous motion of the joint. Further, the sequence of thin layers 72 may advance the integrity of the implant as the thin layers may have better cohesion with less likelihood of separation under flexion and lateral bending.

Referring now to FIG. 11, an implant 80 includes a series of thin layers 82 emanating from a central area 83 and extending outward toward a side wall 84, a top face 85, and a bottom face 86. The layers 82 may be concentric about the nuclear central area 83 and extend generally radially outward toward the side wall 84, top face 85, and bottom face 86. In this embodiment, each successive layer may surround or encapsulate the inner layer such that the implant 80 is layered in multiple directions as compared to the embodiment in FIG. 10 in which the implant 70 is generally laterally layered only in the direction of the side wall 74. The layers 82 may have different moduli of elasticity. For example, the modulus of elasticity of the central area 83 may be relatively low with the moduli of the layers growing increasingly larger toward the outer layers near the side wall 84, the top face 85, and the bottom face 86. In this configuration, the implant 80 would have a soft center and an increasingly stiff periphery. In one embodiment, five or more thin layers may be used.

When the implant 80 is subjected to an asymmetric load, the variance in modulus from the central area 83 and across the layers 82 may allow the central area to shift away from the load, thereby reducing the likelihood that the entire implant will migrate or become expelled from the annulus 22. The thin layers 82 may also affect a relatively smooth, less abrupt, change in moduli which may permit an even, continuous motion of the joint. Further, the sequence of thin layers 82 may advance the integrity of the implant as the thin layers may have better cohesion with less likelihood of separation under flexion and lateral bending.

Referring now to FIG. 12, an implant 90 includes a continuous body 92 having a side wall 94, a top face 95, and a bottom face 96. The body 92 may be formed of a continuous material having a gradual modulus of elasticity gradient which changes from the center of the body 92 outward toward the side wall 94. For example, the modulus of elasticity of the center of the body 92 may be relatively low with the moduli growing increasingly larger toward the side wall 94. In this configuration, the implant 90 would have a soft center and an increasingly stiff periphery.

The body 92 may be formed of an integrally molded elastomeric material with modulus gradation incorporated into the body during the molding process. In this embodiment, the modulus at a given lateral distance between the center and the side wall may be relatively consistent across the span of the body 92 from top face 95 to bottom face 96. Thus, the centers of the top and bottom faces 95, 96 may be relatively soft compared to the portions of the top and bottom faces near the side wall 94.

When the implant 90 is subjected to an asymmetric load, the variance in modulus from the central area 92 to the side wall 94 may allow the central area to shift away from the load, thereby reducing the likelihood that the entire implant will migrate or become expelled from the annulus 22. As compared to the larger regions in the embodiments of FIG. 2 or 4, the gradation of the modulus of elasticity in the body 92 may affect a continuous, less abrupt, change in moduli which may permit smoother, more continuous motion. Further, the continuous body 92 may advance the integrity of the implant 90 as the integrally formed body may be less likely to break apart under flexion and lateral bending.

Referring now to FIG. 13, an implant 100 includes a continuous body 102 having a side wall 104, a top face 105, and a bottom face 106. The body 102 may be formed of a continuous material having a gradual modulus of elasticity gradient which changes from the center of the body 102 outward toward the side wall 104. In this example, the modulus of elasticity of the center of the body 102 may be relatively high with the moduli decreasing toward the side wall 104. In this configuration, the implant 100 would have a stiff center and an increasingly soft periphery.

The body 102 may be formed of an integrally molded elastomeric material with modulus gradation incorporated into the body during the molding process. In this embodiment, the modulus at a given lateral distance between the center and the side wall may be relatively consistent across the span of the body 102 from top face 105 to bottom face 106. As compared to the larger regions in the embodiments of FIG. 2 or 4 or even the layers of FIG. 10, the gradation of the modulus of elasticity in the body 92 may affect a continuous, less abrupt, change in moduli which may permit smoother, more continuous motion. Further, the continuous body 92 may advance the integrity of the implant 90 as the integrally formed body may be less likely to break apart under flexion and lateral bending.

Referring now to FIG. 14, an implant 110 includes a continuous body 112 having a side wall 114, a top face 115, and a bottom face 116. The body 112 may be formed of a continuous material having a gradual modulus of elasticity gradient which changes from the center of the body 112 outward toward the side wall 114, the top face 115, and the bottom face 116. In this example, the modulus of elasticity of the center of the body 112 may be relatively low with the modulus increasing radially outward toward the side wall 104, the top face 115, and the bottom face 116. In this configuration, the implant 110 would have a relatively soft center with an increasingly stiff top, bottom and side periphery.

The body 112 may be formed of an integrally molded elastomeric material with modulus gradation incorporated into the body during the molding process. As compared to the body 92 of FIG. 12, the body 112 may be relatively stiff across the top and bottom faces 115, 116 as well as along the side walls 114.

When the implant 110 is subjected to an asymmetric load, the variance in modulus from the central area 112 to the side wall 114 may allow the central area to shift away from the load, thereby reducing the likelihood that the entire implant will migrate or become expelled from the annulus 22. As compared to the larger regions in the embodiments of FIG. 2 or 4, the gradation of the modulus of elasticity in the body 112 may affect a continuous, less abrupt, change in moduli which may permit smoother, more continuous motion. Further, the continuous body 112 may advance the integrity of the implant 110 as the integrally formed body may be less likely to break apart under flexion and lateral bending.

As shown in FIG. 16, the implant 110 is positioned within the nucleus 21 and within the annulus 22. A portion of the annulus 22 may be removed or otherwise opened to allow entry of the implant 110. This opening may later be sutured, blocked, or otherwise repaired to limit expulsion of the implant 110.

Referring now to FIG. 15, an implant 120 includes a continuous body 122 having a side wall 124, a top face 125, and a bottom face 126. The body 122 may be formed of a continuous material having a gradual modulus of elasticity gradient which changes from the center of the body 122 outward toward the side wall 124, the top face 125, and the bottom face 126. In this example, the modulus of elasticity of the center of the body 122 may be relatively low with the modulus increasing radially outward toward the side wall 124, the top face 125, and the bottom face 126. In this configuration, the implant 120 would have a relatively soft center with an increasingly stiff periphery.

The body 122 may be formed of an integrally molded elastomeric material with modulus gradation incorporated into the body during the molding process. As compared to the body 102 of FIG. 13, the body 122 may be relatively stiff across the top and bottom faces 125, 126 as well as along the side walls 124. As compared to the larger material regions in the embodiments of FIG. 6 or 8 or even the layers of FIG. 11, the gradation of the modulus of elasticity in the body 122 may affect a continuous, less abrupt, change in moduli which may permit smoother, more continuous motion. Further, the continuous body 122 may advance the integrity of the implant 120 as the integrally formed body may be less likely to break apart under flexion and lateral bending.

The gradual changes in gradient may be achieved through molding methods, including injection molding methods. Within an implant formed of an otherwise homogeneous material, the modulus of elasticity may be varied by varying the amount and type of chemical crosslinking. The gradient changes may also result from combining or dispersing additional materials in varying amounts throughout an otherwise homogeneous material to achieve a desired combined or blended modulus. Modulus gradation can also result from the use of reinforcing materials. The implants may be formed of solid materials, for example, molded silicone, hydrogel, or polyurethane. In other embodiments, implants may be more porous, formed, for example, of a woven fabric made of ultra high molecular weight polyethylene (UHMWPE) fibers, polyethylene terephthalate (PET) fibers, polyester fibers, or metallic fibers. The fabric content or weave density may be varied to achieve the gradient change. A woven fabric may also be embedded in a solid polymer material to form an implant having a varied modulus due to fabric concentration, content, or weave density. Furthermore, variations in gradation may be achieved through physical features such as changes in implant thickness, surface patterns, material porosity, or material voids.

The implants described above may be formed of elastomeric materials such as polyurethane, silicone, silicone polyurethane copolymers, polyolefins, such as polyisobutylene rubber and polyisoprene rubber, neoprene rubber, nitrile rubber, vulcanized rubber and combinations thereof. Non-elastic polymers such as polyethylene, polyester, and polyetheretherketone (PEEK) may also be suitable. The non-elastic polymers may be incorporated in the form of fibers, non-woven mesh, woven fabric, or braided structure.

Certain portions of the implant, such as lower modulus regions, layer, or areas may be formed of more deformable or compliant materials including soft elastomers and polymeric gels. Suitable hydrogels may include poly(vinyl alcohol), poly(acrylic acids), poly(methacrylic acids), copolymers of acrylic acid and methacrylic acid, poly(acrylonitrile-acrylic acid), polyacrylamides, poly(N-vinyl-2-pyrrolidone), polyethylene glycol, polyethyleneoxide, polyacrylates, poly(2-hydroxy ethyl methacrylate), copolymers of acrylates with N-vinyl pyrrolidone, N-vinyl lactams, polyurethanes, polyphosphazenes, poly(oxyethylene)-poly(oxypropylene) block polymers, poly(oxyethylene)-poly(oxypropylene) block polymers of ethylene diamine, poly(vinyl acetate), and sulfonated polymers, polysaccharides, proteins, and combinations thereof.

Materials may be selected to achieve a desired performance. For example, in the embodiments of FIGS. 2, 3, 6, 7, the central region may be formed of ultra high molecular weight polyethylene (UHMWPE) and the peripheral region may be formed of polyurethane to achieve a centrally stiffer device. Alternatively, the central region may be formed of silicone with a polyurethane peripheral region. Other suitable material combinations may include a hydrogel central region with silicone peripheral region, a hydrogel central region with polyurethane peripheral region, or a silicone central region with polyurethane peripheral region

In the three region embodiments of FIGS. 4, 5, 8, and 9, the central region may be formed of a gel, the middle region formed of a silicone, and the peripheral region formed of a polyurethane to create a centrally soft and outwardly stiffer device. Alternatively, the central region may be formed of a gel, the middle region formed of a polyurethane, and the peripheral region formed of a silicone to create a device having a stiffer middle region than either the central or peripheral regions. Another suitable material combination may be a hydrogel central region, silicone middle region, and polyurethane peripheral region. Another suitable material combination may be a hydrogel central region, polyurethane middle region, and a woven fabric peripheral region. Another suitable material combination may be a silicone central region, a polyurethane middle region, and a woven fabric peripheral region.

In other alternative embodiments, the implant may have more than two or three regions. For example, a hydrogel center with silicone middle layer, a polyurethane middle layer, and a woven fabric peripheral region.

In the gradient embodiment of FIG. 12, the center of the implant may be formed of 50 Shore A polyurethane with the hardness of the polyurethane gradually increasing to 80 Shore A. In the gradient embodiment of FIG. 14, the center of the implant may be a polyurethane gel that changes from a gelantinous consistency to a harder consistency material such as an 80 Shore A polyurethane. In other alternative gradient embodiments, the hardness may gradually change from 20-100 Shore A or from 40-90 Shore A.

In embodiments in which the central region has a lower modulus of elasticity than the other regions, the higher modulus regions may support and/or contain the softer core. Additionally, under flexion or lateral bending motions, the softer and more deformable central region will deform more than the middle or peripheral regions. Further, as the implant is loaded unevenly or off center, the softer central region may deform and shift away from the load to reduce the potential for the whole implant to displace. This ability to compensate for load shifts may reduce the potential for implant migration or expulsion.

The implants described above may assume any of a variety of shapes including spherical, elliptoid, boomerang, Saturn-like, disc, capsule, kidney, oval, rectangular, or cylindrical. The center regions, layers, or areas of the implants may have a similar or different shape than the overall shape of the implant.

Prior to positioning any of the implants described above in the intervertebral disc space 20, an incision may be made in the annulus fibrosis or an existing annulus defect may be identified. The annulus 22 may be accessed through a posterior, lateral, anterior, or any other suitable approach. In one embodiment, a guide wire or other small instrument may be used to make the initial hole. If necessary, successively larger holes are cut from an initially small puncture. The hole (also called an aperture, an opening, or a portal, for example) may be as small as possible to minimize expulsion of the material through the hole after the surgery is complete.

Also if necessary, a dilator may be used to dilate the hole, making it large enough to deliver the implant to replace or augment the disc nucleus. The dilator may stretch the hole temporarily and avoid tearing so that the hole can return back to its undilated size after the instrument is removed. Although some tearing or permanent stretching may occur, the dilation may be accomplished in a manner that allows the hole to return to a size smaller than its dilated size after the surgery is complete.

Through this annulus opening, all or a portion of the natural nucleus pulposus may be removed. Any of a variety of tools may be used to prepare the disc space, including specialized pituitary rongeurs and curettes for reaching the margins of the nucleus pulposus. Ring curettes may be used to space abrasions from the vertebral endplates as necessary. Using these instruments, a centralized, symmetrical space large enough to accept the implant footprint may be prepared in the disc space. It is understood that the natural nucleus pulposus need not be removed, but rather, as shown in FIG. 16, an implant with modulus variations may be used in cooperation with existing nucleus tissue to compensate for deficiencies in the existing tissue. The disc space may then be distracted to a desired level by distractors or other devices known to the skilled artisan for such purposes. After preparing the disc space 20 and/or annulus 22 for receiving the implant, the implant may be delivered into the intervertebral disc space using any of a variety of techniques known in the art.

As used throughout this description, the terms “modulus” and “modulus of elasticity” are broadly used to refer to physical material properties such as hardness or elasticity. High modulus materials are relatively hard or stiff, and low modulus materials are relatively soft and resilient.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top,” “upper,” “lower,” “bottom,” “left,” “right,” “anterior,” “posterior,” “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

What is claimed is:

1. An intervertebral disc augmentation implant comprising:
an implant body adapted for implantation within an annulus fibrosis the intervertebral disc, adjacent to an at least partially intact nucleus pulposus of the intervertebral disc, and comprising a core area, a peripheral wall area, a top face area, and a bottom face area,
wherein the implant body is formed from at least one material and the implant body has a modulus of elasticity gradient that gradually changes from the core area of the implant body to the peripheral wall area of the implant body.

2. The intervertebral disc augmentation implant of claim 1 wherein the core area of the implant body has a modulus of elasticity lower than the peripheral wall area of the implant body.

3. The intervertebral disc augmentation implant of claim 1 wherein the core area of the implant body has a modulus of elasticity higher than the peripheral wall area of the implant body.

4. The intervertebral disc augmentation implant of claim 1 wherein the implant body has a modulus of elasticity gradient that also gradually changes from the top face area to the bottom face area.

5. The intervertebral disc augmentation implant of claim 4 wherein the top face area has a modulus of elasticity greater than the core area.

6. The intervertebral disc augmentation implant of claim 4 wherein the top face area has a modulus of elasticity lower than the core area.

7. The intervertebral disc augmentation implant of claim 1 wherein the modulus of elasticity gradient changes from a 50 Shore A hardness at the core area to an 80 Shore A hardness at the peripheral wall area.

8. The intervertebral disc augmentation implant of claim 1 wherein the modulus of elasticity gradient changes from a 20 Shore A hardness at the core area to a 100 Shore A hardness at the peripheral wall area.

9. The intervertebral disc augmentation implant of claim 1 wherein the modulus of elasticity gradient changes from a 40 Shore A hardness at the core area to a 90 Shore A hardness at the peripheral wall area.

10. The intervertebral disc augmentation implant of claim 1 wherein the core area has a different shape than the peripheral wall area.

11. The intervertebral disc augmentation implant of claim 1 wherein the peripheral wall area comprises a more highly cross-linked material than the core area.

12. The intervertebral disc augmentation implant of claim 1 wherein the peripheral wall area comprises more reinforcing material than the core area.

13. The intervertebral disc augmentation implant of claim 1 wherein the core area comprises a gel material.

14. The intervertebral disc augmentation implant of claim 1 wherein the core area comprises a silicone material.

15. The intervertebral disc augmentation implant of claim 1 wherein the peripheral wall area comprises a polyurethane material.

16. The intervertebral disc augmentation implant of claim 1 wherein the implant body is formed of at least two materials with a first material dispersed in gradually varying density within a second material.

17. The intervertebral disc augmentation implant of claim 1 wherein the core area and the peripheral wall area are both formed of silicone.

18. The intervertebral disc augmentation implant of claim 1 wherein the core area and the peripheral wall area are both formed of hydrogel.

19. The intervertebral disc augmentation implant of claim 1 wherein the implant body comprises a woven fabric.

20. The intervertebral disc augmentation implant of claim 19 wherein the woven fabric comprises UHMWPE fibers.

21. The intervertebral disc augmentation implant of claim 19 wherein the woven fabric comprises PET fibers.

22. The intervertebral disc augmentation implant of claim 19 wherein the woven fabric comprises metallic fibers.

23. The intervertebral disc augmentation implant of claim 19 wherein the woven fabric varies to create the modulus of elasticity gradient.

24. The intervertebral disc augmentation implant of claim 19 wherein the woven fabric is embedded in a solid polymer material.

25. A method of augmenting a nucleus pulposus comprising:
accessing an annulus surrounding the nucleus pulposus;
forming an opening in the annulus; and
inserting an intervertebral nucleus augmentation implant wherein the implant comprises
an implant body including a core area, a peripheral wall area, a top face, and a bottom face and further wherein the implant body is formed from at least one material and the implant body has a modulus of elasticity gradation that gradually changes from the core area of the implant body to the peripheral wall area of the implant body.

26. The method of claim 25 further comprising:
removing at least a portion of the nucleus pulposus.

27. The method of claim 25 wherein the step of inserting further comprises placing the intervertebral nucleus augmentation implant in contact with at least a portion of the nucleus pulposus.

28. An implant for augmenting or replacing the natural nucleus pulposus within an intervertebral space, the implant comprising:
a central region and
an outer region extending between the central region and an outer wall, wherein the outer region has a modulus of elasticity that becomes progressively higher from the central region to the outer wall,
wherein the implant is sized for insertion through an opening in an annulus fibrosis surrounding the nucleus pulposus.

29. The implant of claim 28 wherein the outer region comprises a plurality of layers having progressively higher moduli of elasticity.

30. The implant of claim 29 wherein the plurality of layers encase the central region.

31. The implant of claim 29 wherein the plurality of layers includes at least five layers.

32. The implant of claim 28 wherein the outer region comprises a continuous material having a modulus of elasticity gradient that gradually increases from the central region to the outer wall.

33. The implant of claim 28 wherein the outer wall has a side surface, a top surface, and a bottom surface and wherein the outer region has a modulus of elasticity that becomes progressively higher from the central region to the top surface and from the central region to the bottom surface.

34. The implant of claim 28 wherein the outer wall has a side surface, a top surface, and a bottom surface and wherein the outer region has a modulus of elasticity that becomes progressively higher from the central region to the side surface.
